(19) United States
(12) Patent Application Publication
(10) Pub. No.: US 2022/0347397 A1
(43) Pub. Date: Nov. 3, 2022

(54) NON-REUSABLE SYRINGE AND METHODS OF USE

(71) Applicant: KEVIN SULLIVAN, PATCHOGUE, NY (US)

(72) Inventor: KEVIN SULLIVAN, PATCHOGUE, NY (US)

(21) Appl. No.: 17/244,088

(22) Filed: Apr. 29, 2021

(51) Int. Cl. A61M 5/50; A61M 5/31

(52) U.S. Cl. CPC A61M 5/502; A61M 5/3129; A61M 2005/5033

(57) ABSTRACT

A syringe includes a barrel extending along a longitudinal axis between opposite proximal and distal ends. The barrel includes a proximal inner surface and a distal inner surface. The proximal inner surface defines a proximal chamber and the distal inner surface defines a distal chamber. The barrel includes first teeth extending from the proximal inner surface. A piston includes a body having opposite proximal and distal sections. The piston includes second teeth extending from an outer surface of the proximal section. The second teeth directly engage the first teeth to allow the piston to translate distally relative to the barrel along the longitudinal axis and prevent the piston from translating proximally relative to the barrel along the longitudinal axis. Systems, kits and methods are disclosed.

### TECHNICAL FIELD

The present disclosure generally relates to syringes, and more particularly to devices, non-reusable syringes configured for injections, such as, for example, hypodermic injections.

### BACKGROUND

Even when used medically, the reuse of syringes increases the transmission of various types of diseases. Additional risks entail when syringes are reused by intravenous drug abusers. Accordingly, many attempts have been made to provide syringes that cannot be reused. However, many syringes that were designed not to be reused, have proven ineffective. For example, some syringes that were designed not to be reused have safety features that can easily be removed and/or circumvented to allow the syringes to be reused. Other syringes that were designed not to be reused were unable to be properly used in a first instance to dispense a pre-loaded amount of biologic, for example. This disclosure describes improvements over these prior art technologies.

### SUMMARY

In one embodiment, in accordance with the principles of the present disclosure, a syringe comprises a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises first teeth extending from the proximal inner surface. A piston comprises a body having opposite proximal and distal sections. The piston comprises second teeth extending from an outer surface of the proximal section. The second teeth directly engage the first teeth to allow the piston to translate distally relative to the barrel along the longitudinal axis and prevent the piston from translating proximally relative to the barrel along the longitudinal axis.

In some embodiments, the first teeth each include a trailing edge that faces the distal end and a leading edge that faces away from the distal end, the leading edges each extending at an acute angle relative to the longitudinal axis, the trailing edges each extending perpendicular to the longitudinal axis.

In some embodiments, the body extends along a second longitudinal axis between a proximal end that includes the proximal section and a distal end that includes the distal section. The second teeth each include a trailing edge that faces the proximal end of the body and a leading edge that faces away from the proximal end of the body. The leading edges each extend at an acute angle relative to the second longitudinal axis. The trailing edges each extend perpendicular to the second longitudinal axis.

In some embodiments, the first teeth each include a trailing edge that faces the distal end and a leading edge that faces away from the distal end. The leading edges each extend at an acute angle relative to the longitudinal axis. The trailing edges each extend perpendicular to the longitudinal axis. The body extends along a second longitudinal axis between a proximal end that includes the proximal section and a distal end that includes the distal section. The second teeth each include a trailing edge that faces the proximal end of the body and a leading edge that faces away from the proximal end of the body. The leading edges of the second teeth each extend at an acute angle relative to the second longitudinal axis. The trailing edges of the second teeth each extend perpendicular to the second longitudinal axis. In some embodiments, the acute angles are the same. In some embodiments, the acute angles are different.

In some embodiments, the first teeth each include a trailing edge that faces the distal end, a leading edge that faces away from the distal end and an intermediate edge between the trailing edge and the leading edge. The leading edges each extend at an acute angle relative to the longitudinal axis. The trailing edges each extend perpendicular to the longitudinal axis. The intermediate edges each extend parallel to the longitudinal axis.

In some embodiments, the body extends along a second longitudinal axis between a proximal end that includes the proximal section and a distal end that includes the distal section. The second teeth each include a trailing edge that faces the proximal end of the body, a leading edge that faces away from the proximal end of the body and an intermediate edge between the leading edge and the trailing edge. The leading edges each extend at an acute angle relative to the second longitudinal axis. The trailing edges each extend perpendicular to the second longitudinal axis. The intermediate edges each extend parallel to the second longitudinal axis.

In some embodiments, the first teeth each extend 360 degrees about the longitudinal axis.

In some embodiments, the second teeth each extend 360 degrees about a longitudinal axis defined by the body.

In some embodiments, the first teeth each include a pocket that is filled with epoxy, the pockets being configured to break as the second teeth move over the first teeth.

In some embodiments, the second teeth each include a pocket that is filled with epoxy, the pockets being configured to break as the first teeth move over the second teeth.

In some embodiments, the barrel includes spaced apart first and second rails extending from the distal inner surface. The body comprises spaced apart first and second grooves extending into an outer surface of the distal section. The rails are each disposed in one of the grooves when the second teeth engage the first teeth.

In one embodiment, in accordance with the principles of the present disclosure, a syringe includes a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises first teeth extending from the distal inner surface. A piston comprises a body having opposite proximal and distal sections. The piston comprises second teeth extending from an outer surface of the distal section. The second teeth directly engage the first teeth to allow the piston to translate distally relative to the barrel along the longitudinal axis and prevent the piston from translating proximally relative to the barrel along the longitudinal axis.

In some embodiments, the first teeth each include a trailing edge that faces the distal end and a leading edge that faces away from the distal end. The leading edges each extend at an acute angle relative to the longitudinal axis. The trailing edges each extend perpendicular to the longitudinal axis. The body extends along a second longitudinal axis between a proximal end that includes the proximal section and a distal end that includes the distal section. The second teeth each include a trailing edge that faces the proximal end of the body and a leading edge that faces away from the proximal end of the body. The leading edges of the second teeth each extend at an acute angle relative to the second longitudinal axis. The trailing edges of the second teeth each extend perpendicular to the second longitudinal axis.

In some embodiments, the first teeth each include a trailing edge that faces the distal end, a leading edge that faces away from the distal end and an intermediate edge between the trailing edge and the leading edge. The leading edges of the first teeth each extend at an acute angle relative to the longitudinal axis. The trailing edges of the first teeth each extend perpendicular to the longitudinal axis. The intermediate edges each extend parallel to the longitudinal axis.

In some embodiments, the body extends along a second longitudinal axis between a proximal end that includes the proximal section and a distal end that includes the distal section. The second teeth each include a trailing edge that faces the proximal end of the body, a leading edge that faces away from the proximal end of the body and an intermediate edge between the leading edge and the trailing edge. The leading edges each extend at an acute angle relative to the second longitudinal axis. The trailing edges each extend perpendicular to the second longitudinal axis. The intermediate edges each extend parallel to the second longitudinal axis.

In some embodiments, the first teeth each extend 360 degrees about the longitudinal axis.

In some embodiments, the second teeth each extend 360 degrees about a longitudinal axis defined by the body.

In one embodiment, in accordance with the principles of the present disclosure, a syringe includes a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises first teeth extending from the proximal inner surface. The first teeth extend 360 degrees about the longitudinal axis. The barrel includes spaced apart first and second rails extending from the distal inner surface. A piston comprises a body having opposite proximal and distal sections. The piston comprises second teeth extending from an outer surface of the proximal section. The second teeth directly engage the first teeth to allow the piston to translate distally relative to the barrel along the longitudinal axis and prevent the piston from translating proximally relative to the barrel along the longitudinal axis. The body comprises spaced apart first and second grooves extending into an outer surface of the distal section. The rails are each disposed in one of the grooves when the second teeth engage the first teeth. The first teeth each include a trailing edge that faces the distal end and a leading edge that faces away from the distal end. The leading edges each extend at an acute angle relative to the longitudinal axis. The trailing edges each extend perpendicular to the longitudinal axis. The body extends along a second longitudinal axis between a proximal end that includes the proximal section and a distal end that includes the distal section. The second teeth each extending 360 degrees about the second longitudinal axis. The second teeth each include a trailing edge that faces the proximal end of the body and a leading edge that faces away from the proximal end of the body. The leading edges of the second teeth each extend at an acute angle relative to the second longitudinal axis. The trailing edges of the second teeth each extend perpendicular to the second longitudinal axis. The first teeth each include an intermediate edge between the trailing edges of the first teeth and the leading edges of the first teeth. The intermediate edges of the first teeth each extend parallel to the longitudinal axis. The second teeth each include an intermediate edge between the leading edges of the second teeth and the trailing edges of the second teeth. The intermediate edges of the second teeth each extend parallel to the second longitudinal axis.

In one embodiment, in accordance with the principles of the present disclosure, a syringe includes a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises a first flange extending from the proximal inner surface. A piston comprises a body having opposite proximal and distal sections. The piston comprises a second flange extending from an outer surface of the proximal section. The piston is configured for engagement with the barrel such that the distal section is positioned in the distal chamber, the proximal section is positioned in the proximal chamber and the second flange is positioned between the first flange and the distal end to prevent the piston from translating proximally relative to the barrel along the longitudinal axis.

In some embodiments, the first flange includes an end surface extending at a first angle relative to the longitudinal axis and the second flange includes an end surface extending at a second angle relative to the longitudinal axis. In some embodiments, the first angle is between about 1 degree and about 80 degrees and the second angle is between about −1 degree and about −80 degrees. In some embodiments, the first angle is between about 30 degrees and about 60 degrees and the second angle is between about −30 degrees and about −60 degrees.

In some embodiments, a distal surface of the first flange directly engages a proximal surface of the second flange when the distal section is positioned in the distal chamber, the proximal section is positioned in the proximal chamber and the second flange is positioned between the first flange and the distal end. In some embodiments, the distal surface and the proximal surface each extend perpendicular to the longitudinal axis. In some embodiments, the first flange includes an end surface extending at a first angle relative to the longitudinal axis and the second flange includes an end surface extending at a second angle relative to the longitudinal axis. In some embodiments, the first angle is between about 30 degrees and about 60 degrees and the second angle is between about −30 degrees and about −60 degrees. In some embodiments, the barrel includes first and second arms extending in opposite from the proximal end and the piston comprises a ledge extending from the proximal section, a distal surface of the ledge engaging proximal surfaces of the arms when the distal surface of the first flange directly engages the proximal surface of the second flange. In some embodiments, the first flange is positioned between the arms and the distal end and the second flange is positioned between the ledge and the distal section. In some embodiments, the arms and the ledge each extend perpendicular to the longitudinal axis.

In some embodiments, the first flange defines a circular opening having a first diameter and the second flange has a second diameter. In some embodiments, the first diameter is equal to the second diameter. In some embodiments, the first diameter is greater than the second diameter. In some embodiments, the first diameter is less than the second diameter.

In one embodiment, in accordance with the principles of the present disclosure, a syringe includes a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises a first flange extending from the proximal inner surface. The first flange comprises opposite proximal and distal surfaces and a first end surface extending from the proximal surface of the first flange to the distal surface of the first flange. A piston comprises a body having opposite proximal and distal sections. The piston comprises a second flange extending from an outer surface of the proximal section. The second flange comprises opposite proximal and distal surfaces and a second end surface extending from the proximal surface of the second flange to the distal surface of the first second flange. The piston is configured for engagement with the barrel to move the syringe from a first configuration in which the first flange is positioned between the second flange and the distal to a second configuration in which the second flange is positioned between the first flange and the distal end.

In some embodiments, the piston is prevented from translating proximally relative to the barrel along the longitudinal axis when the syringe is in the second configuration.

In some embodiments, the distal surface of the first flange directly engages the proximal surface of the second flange when the syringe is in the second configuration to prevent the piston from translating proximally relative to the barrel along the longitudinal axis.

In some embodiments, the second end surface slides along the first end surface as the syringe moves from the first configuration to the second configuration

In one embodiment, in accordance with the principles of the present disclosure, a syringe includes a barrel extending along a longitudinal axis between opposite proximal and distal ends. The barrel comprises a proximal inner surface and a distal inner surface. The proximal inner surface defines a proximal chamber and the distal inner surface defines a distal chamber. The barrel comprises a first flange extending from the proximal inner surface. The first flange comprises opposite proximal and distal surfaces and a first end surface extending from the proximal surface of the first flange to the distal surface of the first flange. A piston comprises a body having opposite proximal and distal sections. The piston comprises a second flange extending from an outer surface of the proximal section. The second flange comprises opposite proximal and distal surfaces and a second end surface extending from the proximal surface of the second flange to the distal surface of the first second flange. The piston is configured for engagement with the barrel to move the syringe from a first configuration in which the first flange is positioned between the second flange and the distal to a second configuration in which the second flange is positioned between the first flange and the distal end. The distal surface of the first flange directly engages the proximal surface of the second flange when the syringe is in the second configuration to prevent the piston from translating proximally relative to the barrel along the longitudinal axis. The second end surface slides along the first end surface as the syringe moves from the first configuration to the second configuration. The first end surface extends at a first angle relative to the longitudinal axis and the second end surface extends at a second angle relative to the longitudinal axis. The first angle is between about 30 degrees and about 60 degrees and the second angle is between about −30 degrees and about −60 degrees. The barrel includes first and second arms extending in opposite from the proximal end and the piston comprises a ledge extending from the proximal section. A distal surface of the ledge engages proximal surfaces of the arms when the distal surface of the first flange directly engages the proximal surface of the second flange. The arms and the ledge each extend perpendicular to the longitudinal axis. The first flange defines a circular opening having a first diameter and the second flange has a second diameter.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

The present disclosure will become more readily apparent from the specific description accompanied by the following drawings, in which:

FIG. 1 is a side, cross-sectional view of one embodiment of a syringe in accordance with the principles of the present disclosure;

FIG. 2 is a side, cross-sectional view of a component of the syringe shown in FIG. 1;

FIG. 2A is a side, cross-sectional view of a portion of the component shown in FIG. 2;

FIG. 3 is a side, cross-sectional view of one embodiment of a portion of the component shown in FIG. 2 at Detail A in FIG. 2;

FIG. 4 is a side, cross-sectional view of one embodiment of a portion of the component shown in FIG. 2 at Detail A in FIG. 2;

FIG. 5 is a side, cross-sectional view of a component of the syringe shown in FIG. 1;

FIG. 5A is a side, cross-sectional view of a portion of the component shown in FIG. 5;

FIG. 6 is a side, cross-sectional view of one embodiment of a portion of the component shown in FIG. 5 at Detail B in FIG. 5;

FIG. 7 is a side, cross-sectional view of one embodiment of a portion of the component shown in FIG. 3 and the component shown in FIG. 6;

FIG. 8 is a side, cross-sectional view of one embodiment of a portion of the component shown in FIG. 4 and the component shown in FIG. 6;

FIG. 9 is a side, cross-sectional view of one embodiment of a portion of the component shown in FIG. 5 at Detail B in FIG. 5;

FIG. 10 is a side, cross-sectional view of one embodiment of a portion of the component shown in FIG. 3 and the component shown in FIG. 9;

FIG. 11 is a side, cross-sectional view of one embodiment of a portion of the component shown in FIG. 4 and the component shown in FIG. 9;

FIG. 12A is a top, cross-sectional view of one embodiment of a portion of the component shown in FIG. 2 between lines C and D in FIG. 2;

FIG. 12B is a top, cross-sectional view of one embodiment of a portion of the component shown in FIG. 5 between lines E and F in FIG. 5;

FIG. 12C is a top, cross-sectional view of one embodiment of a portion of the component shown in FIG. 12A and the component in FIG. 12B;

FIG. 13A is a top, cross-sectional view of one embodiment of a portion of the component shown in FIG. 2 between lines C and D in FIG. 2;

FIG. 13B is a top, cross-sectional view of one embodiment of a portion of the component shown in FIG. 5 between lines E and F in FIG. 5;

FIG. 13C is a top, cross-sectional view of one embodiment of a portion of the component shown in FIG. 12A and the component in FIG. 12B;

FIG. 14 is a side, cross-sectional view of the component shown in FIG. 5 being inserted into the component shown in FIG. 2;

FIG. 15 is a side, cross-sectional view of the component shown in FIG. 5 fully inserted into the component shown in FIG. 2;

FIG. 16 is a side, cross-sectional view of one embodiment of a syringe in accordance with the principles of the present disclosure;

FIG. 17 is a side, cross-sectional view of a component of the syringe shown in FIG. 16;

FIG. 18 is a side, cross-sectional view of a component of the syringe shown in FIG. 16;

FIG. 19 is a side, cross-sectional view of the component shown in FIG. 18 fully inserted into the component shown in FIG. 17;

FIG. 20 is a top, cross-sectional view of one embodiment of a portion of the component shown in FIG. 17 between lines G and H in FIG. 17;

FIG. 21 is a top, cross-sectional view of one embodiment of a portion of the component shown in FIG. 18 between lines I and J in FIG. 18;

FIG. 22 is a side, cross-sectional view of one embodiment of a syringe in accordance with the principles of the present disclosure;

FIG. 23 is a side, cross-sectional view of a component of the syringe shown in FIG. 22;

FIG. 24 is a side, cross-sectional view of a component of the syringe shown in FIG. 22;

FIG. 25 is a side, cross-sectional view of a portion of the component shown in FIG. 23;

FIG. 26 is a side, cross-sectional view of a portion of the component shown in FIG. 24;

FIG. 27 is a side, cross-sectional view of a portion of the component shown in FIG. 23;

FIG. 28 is a side, cross-sectional view of a portion of the component shown in FIG. 24;

FIG. 29 is a top, cross-sectional view of one embodiment of a portion of the component shown in FIG. 23 between lines K and L in FIG. 23;

FIG. 30 is a top, cross-sectional view of one embodiment of a portion of the component shown in FIG. 24 between lines M and N in FIG. 24;

FIG. 31 is a side, cross-sectional view of a portion of the component shown in FIG. 24;

FIG. 32 is a side, cross-sectional view of one embodiment of a syringe in accordance with the principles of the present disclosure;

FIG. 33 is a side, cross-sectional view of a component of the syringe shown in FIG. 32;

FIG. 34 is a side, cross-sectional view of a component of the syringe shown in FIG. 32;

FIG. 35 is a side, cross-sectional view of a portion of the component shown in FIG. 33;

FIG. 36 is a side, cross-sectional view of a portion of the component shown in FIG. 34;

FIG. 37 is a top, cross-sectional view of one embodiment of a portion of the component shown in FIG. 33; and

FIG. 38 is a top, cross-sectional view of one embodiment of a portion of the component shown in FIG. 34.

### DETAILED DESCRIPTION

The present disclosure generally relates to a dispensing system, and more particularly to a dispensing system that includes one or more syringes that prevent reuse of the syringe. In some embodiments, the syringes are pre-filled with a material, such as, for example, a biologic. In some embodiments, the syringes are not pre-filled with a material, such as, for example, a biologic. In some embodiments, the syringes each include a barrel and a plunger, wherein the plunger is rotatable relative to the barrel when the plunger is disposed in the barrel. In some embodiments, the syringes each include a barrel and a piston, wherein the plunger is prevented from rotating relative to the barrel when the piston is disposed in the barrel. In some embodiments, a needle, such as, for example, a hypodermic needle is coupled to a distal end of the barrel. In some embodiments, the needle is integrally and/or monolithically formed with the barrel such that the needle cannot be removed from the barrel without damaging the barrel and/or the needle.

In some embodiments, the barrel includes first teeth and the plunger includes second teeth that engage the first teeth. In some embodiments, the first teeth each extend parallel to one another and the second teeth each extend parallel to one another. In some embodiments, the first teeth each extend 360 degrees about a longitudinal axis defined by the barrel and the second teeth each extend 360 degrees about a longitudinal axis defined by the plunger. In some embodiments, the first teeth each include an edge that extends perpendicular to a longitudinal axis defined by the barrel and the second teeth each include an edge that extends perpendicular to a longitudinal axis defined by the plunger.

In some embodiments, the piston includes a body and a plunger that is coupled to the body. In some embodiments, the plunger is integrally and/or monolithically formed with the body of the piston. In some embodiments, the plunger cannot be removed from the body of the piston without damaging the plunger and/or the body of the piston. In some embodiments, the piston includes a gasket, such as, for example, an O-ring positioned about the body of the piston, wherein the gasket is configured to form a waterproof and/or airproof seal with an inner surface of the barrel when the piston is positioned in the barrel.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

It is noted that, as used in this specification and the appended claims, the singular forms “a,” “an,” and “the,” include plural referents unless expressly and unequivocally limited to one referent. Thus, for example, reference to “a flange” or a “device” includes one, two, three or more flanges or one, two, three or more devices.

This disclosure is directed to a dispensing system 40. In some embodiments, the components of dispensing system 40 can be fabricated from biologically acceptable materials suitable for medical applications, including metals, synthetic polymers, ceramics and bone material and/or their composites, depending on the particular application and/or preference of a medical practitioner. For example, the components of dispensing system 40, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, and their combinations.

Various components of dispensing system 40 may have material composites, including the above materials, to achieve various desired characteristics such as strength, rigidity, elasticity, compliance, biomechanical performance, durability and radiolucency or imaging preference. The components of dispensing system 40, individually or collectively, may also be fabricated from a heterogeneous material such as a combination of two or more of the above-described materials. The components of dispensing system 40 may be monolithically formed, integrally connected or comprise fastening elements and/or instruments, as described herein.

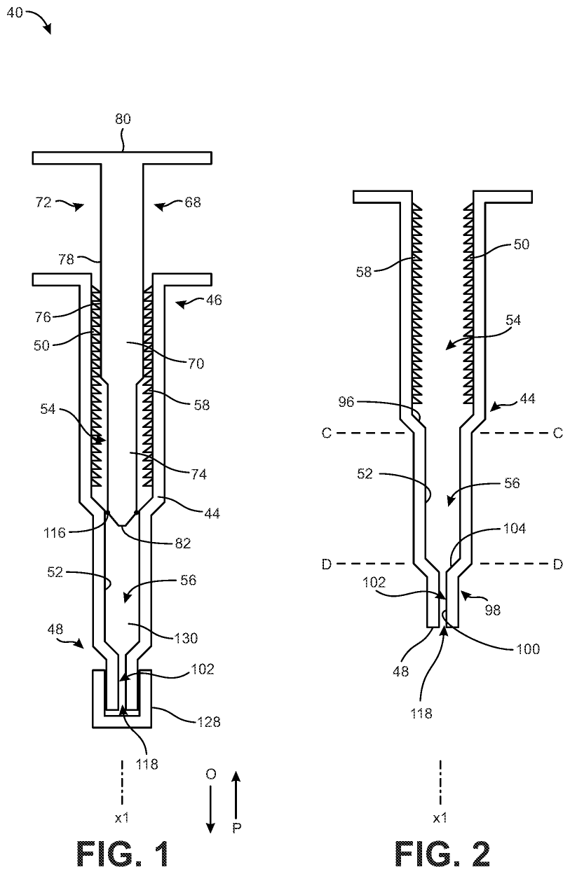

Dispensing system 40 includes a syringe 42, as shown in FIGS. 1-15. Syringe 42 includes a barrel 44 extending along a longitudinal axis X1 between a proximal end 46 and an opposite distal end 48. Barrel 44 comprises a proximal inner surface 50 and a distal inner surface 52. Proximal inner surface 50 defines a proximal chamber 54 and distal inner surface 52 defines a distal chamber 56. Chamber 56 is in communication with chamber 54. In some embodiments, surface 52 is continuous with surface 50. That is, there are no gaps or recesses between surface 52 and surface 50. Barrel 44 comprises a plurality of first teeth 58 extending outwardly from proximal inner surface 50. Teeth 58 are disposed in a serial orientation along axis X1 (a length of barrel 44). That is, teeth 58 are non-helical. In some embodiments, first teeth 58 each extend 360 degrees about longitudinal axis X1. In some embodiments, surface 52 and teeth 58 define a gear rack similar to a gear rack of a cable tie, for example. In some embodiments, barrel 44 is monolithic. In some embodiments, chamber 54 and/or chamber 56 may have various cross section configurations, such as, for example, oval, oblong, triangular, rectangular, square, polygonal, irregular, uniform, non-uniform, variable, tubular and/or tapered.

Teeth 58 each include a trailing edge 60 that faces distal end 48 and a leading edge 62 that faces away from distal end 48. Edges 60, 62 each extend directly from surface 50. Leading edges 62 each extend at an angle α relative to longitudinal axis X1. In some embodiments, angle α is an acute angle. In some embodiments, angle α is between about 5 degrees and about 85 degrees. In some embodiments, angle α is between about 15 degrees and about 75 degrees. In some embodiments, angle α is between about 25 degrees and about 65 degrees. In some embodiments, angle α is between about 35 degrees and about 55 degrees. In some embodiments, angle α is about 45 degrees. In some embodiments, trailing edges 60 each extend perpendicular to longitudinal axis X1. In some embodiments, edges 60, 62 converge at a point 64, as shown in FIGS. 3 and 10, for example. That is, edges 60, 62 each have a first end that extends directly from surface 50 and an opposite second end that defines a portion of point 64. In some embodiments, teeth 58 each include an intermediate edge 66 that extends from trailing edge 60 to leading edge 62, as shown in FIGS. 4, 8 and 9, for example. That is, edges 60, 62 each have a first end that extends directly from surface 50 and an opposite second end that terminates at intermediate edge 66. In some embodiments, intermediate edges 66 each extend parallel to longitudinal axis X1. In some embodiments, intermediate edges 66 each extend at an acute angle to longitudinal axis X1. In some embodiments, at least one of edges 62, 64, 66 is entirely planar. In some embodiments, teeth 58 each extend 360 degrees about longitudinal axis X1 in a plane that extends perpendicular to longitudinal axis X1. In such embodiments, leading edges 62 each extend at angle α 360 degrees about axis X1 in a plane that extends perpendicular to axis X1, trailing edges 60 each extend perpendicular to axis X1 360 degrees about axis X1 in a plane that extends perpendicular to axis X1 and/or intermediate edges 66 each extend parallel to axis X1 360 degrees about axis X1 in a plane that extends perpendicular to longitudinal axis X1.

A plunger, such as, for example, a piston 68 comprises a body 70 having a proximal section 72 and an opposite distal section 74. Section 72 is configured for disposal in chamber 54 and section 74 is configured for disposal in chamber 56, as discussed herein. Piston 68 comprises second teeth 76 extending from an outer surface 78 of proximal section 72. Teeth 76 are configured to directly engage first teeth 58 to allow piston 68 to translate distally relative to barrel 44 along axis X1 and prevent piston 68 from translating proximally relative to barrel 44 along axis X1. Body 70 extends along a second longitudinal axis X2 between a proximal end 80 that includes proximal section 72 and a distal end 82 that includes distal section 74. Teeth 76 each extend 360 degrees about axis X2. Teeth 76 are disposed in a serial orientation along axis X2 (a length of piston 68). That is, teeth 76 are non-helical. In some embodiments, teeth 76 each extend 360 degrees about longitudinal axis X2. In some embodiments, teeth 76 define ratchets or gears similar to a pawl or ratchet of a cable tie, for example, and are configured for engagement with the gear rack defined by surface 50 and teeth 58 to allow piston 68 to translate distally relative to barrel 44 along axis X1 and prevent piston 68 from translating proximally relative to barrel 44 along axis X1, as discussed herein. In some embodiments, piston 68 is monolithic.

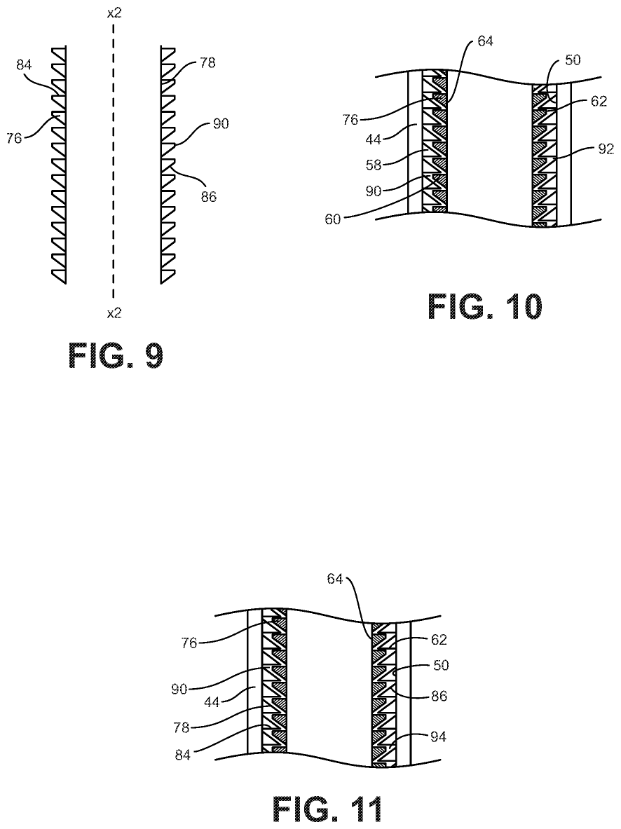

Teeth 76 each include a trailing edge 84 that faces end 80 and a leading edge 86 that faces away from end 80 and toward end 82. Edges 84, 86 each extend directly from surface 78. Leading edges 86 each extend at an angle β relative to longitudinal axis X2. In some embodiments, angle β is an acute angle. In some embodiments, angle β is equal to angle α. In some embodiments, angle β is different than angle α. In some embodiments, angle β is less than angle α. In some embodiments, angle β is greater than angle α. In some embodiments, angle β is between about 5 degrees and about 85 degrees. In some embodiments, angle β is between about 15 degrees and about 75 degrees. In some embodiments, angle β is between about 25 degrees and about 65 degrees. In some embodiments, angle β is between about 35 degrees and about 55 degrees. In some embodiments, angle β is about 45 degrees. In some embodiments, trailing edges 84 each extend perpendicular to longitudinal axis X2. In some embodiments, edges 84, 86 converge at a point 88, as shown in FIGS. 6 and 8, for example. That is, edges 84, 86 each have a first end that extends directly from surface 78 and an opposite second end that defines a portion of point 88. In some embodiments, teeth 76 each include an intermediate edge 90 that extends from trailing edge 84 to leading edge 86, as shown in FIGS. 9-11, for example. That is, edges 84, 86 each have a first end that extends directly from surface 78 and an opposite second end that terminates at intermediate edge 90. In some embodiments, intermediate edges 90 each extend parallel to longitudinal axis X2. In some embodiments, intermediate edges 90 each extend at an acute angle to longitudinal axis X2. In some embodiments, at least one of edges 84, 86, 90 is entirely planar. In some embodiments, teeth 76 each extend 360 degrees about longitudinal axis X2 in a plane that extends perpendicular to longitudinal axis X2. In such embodiments, leading edges 86 each extend at angle β 360 degrees about axis X2 in a plane that extends perpendicular to axis X2, trailing edges 84 each extend perpendicular to axis X2 360 degrees about axis X2 in a plane that extends perpendicular to longitudinal axis X1 and/or intermediate edges 90 each extend parallel to axis X2 360 degrees about axis X2 in a plane that extends perpendicular to longitudinal axis X1.

In some embodiments, teeth 76 engage teeth 58 such that points 88 are each positioned between edges 60, 62 of adjacent teeth 58 and points 64 are positioned between edges 84, 86 of adjacent teeth 76, as shown in FIG. 7, for example. In some embodiments, teeth 76 completely fill the space between adjacent teeth 58 and teeth 58 completely fill the space between adjacent teeth 76. That is, when teeth 76 engage teeth 58, there is no space between adjacent teeth 58 and no space between adjacent teeth 76.

In some embodiments, teeth 76 engage teeth 58 such that points 88 are each positioned between edges 60, 62 of adjacent teeth 58 and edges 90 are positioned between adjacent teeth 76, as shown in FIG. 8, for example. In some embodiments, edges 90 directly engage surface 78 between adjacent teeth 76. In some embodiments, teeth 76 completely fill the space between adjacent teeth 58 and teeth 58 completely fill the space between adjacent teeth 76. That is, when teeth 76 engage teeth 58, there is no space between adjacent teeth 58 and no space between adjacent teeth 76.

In some embodiments, teeth 76 engage teeth 58 such that edges 90 are each positioned between edges 60, 62 of adjacent teeth 58 and points 64 are positioned between adjacent teeth 76 to define gaps 92 between edges 90 and surface 50, as shown in FIG. 10, for example.

In some embodiments, teeth 76 engage teeth 58 such that edges 90 are each positioned between edges 60, 62 of adjacent teeth 58 and edges 66 are positioned between edges 84, 86 of adjacent teeth 76 to define gaps 94 between edges 90 and surface 50, as shown in FIG. 11, for example. In some embodiments, edges 66 directly engage surface 78 between adjacent teeth 76.

In some embodiments, barrel 44 includes an inclined inner surface 96 extending continuously from surface 50 to surface 52. Surface 96 extends at an acute angle relative to axis X1. In some embodiments, barrel 44 includes a nozzle 98. Nozzle 98 includes an inner surface 100 that defines a channel 102. Channel 102 is in communication with chamber 56. Barrel 44 includes an inclined inner surface 104 extending continuously from surface 52 to surface 100. Surface 104 extends at an acute angle relative to axis X1. Piston 68 includes an inclined outer surface 106 extending continuously from surface 78 to an outer surface 108 of section 74. Surface 106 extends at an acute angle relative to axis X2. Piston 68 includes a distal tip 100 having an inclined outer surface 112. Surface 112 extends at an acute angle relative to axis X2. Piston 68 is configured to be inserted into barrel 44 such that axis X2 is coaxial with axis X1. When piston 68 is fully inserted into barrel 44, surface 112 directly engages surface 104 and surface 106 directly engages surface 96.

In some embodiments, surface 52 is smooth and/or even along an entire length of surface 52. That is, surface 52 is free of any cavities or recesses that extend into surface 52 and is also free of any projections or extensions that extend from surface 52. Piston 68 includes a groove 114 extending into surface 108 and a gasket, such as, for example, an O-ring 116 positioned in groove 114. O-ring 116 is configured to engage and slide along surface 52 when piston 68 is being inserted into barrel 44 such that O-ring 116 forms an airtight and/or watertight seal with surface 52. This allows section 74 to push any material, such as, for example, a liquid within chamber 56 and/or channel 102 out of syringe 42 through an opening 118 in distal end of nozzle 98. In some embodiments, O-ring 116 is made from an elastomeric material.

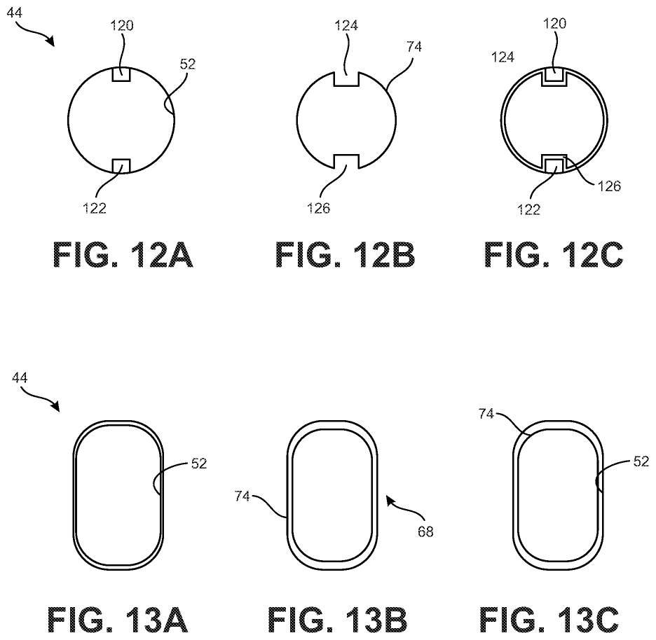

In some embodiments, chamber 54, chamber 56, section 72 and section 74 each have a circular cross-sectional configuration such that piston 68 is rotatable relative to barrel 44 about axis X1. In some embodiments, piston 68 is prevented from rotating relative to barrel 44 about axis X1. For example, in some embodiments, barrel 44 includes spaced apart first and second rails 120, 122 extending from inner surface 52 and section 74 includes spaced apart grooves 124, 126 extending into outer surface 108. Rails 120, 122 each extend parallel to axis X1 and grooves 124, 126 each extend parallel to axis X2. Rail 120 is disposed in groove 124 and rail 122 is disposed in groove 126 as piston 68 is inserted into barrel 44, as shown in FIG. 12C, to prevent piston 68 from rotating relative to barrel 44 about axis X1. That is, rails 120, 122 are disposed in grooves 124, 126 when second teeth 76 engage first teeth 58. In some embodiments, a portion of barrel 44 that includes chamber 52 includes a non-circular cross-sectional configuration, as shown in FIG. 13A, and section 74 of piston 68 includes a non-circular cross-sectional configuration, as shown in FIG. 13B, to prevent piston 68 from rotating relative to barrel 44 about axis X1 when section 74 is inserted into chamber 56.

In operation and use, system 40 includes a material 130 and syringe 42 may be used to mix and/or dispense material 130. In some embodiments, material 130 comprises a material, such as, for example, a liquid, gel, paste, cement, gum, ointment, cream and/or foam. In some embodiments, material 130 comprises one or more biologics, such as, for example, one or more drugs. In some embodiments, material 130 is pre-loaded into at least one of chamber 54, chamber 56 and channel 102. In some embodiments, a cap 128 is coupled to nozzle 98 to maintain material 130 in chamber 54, chamber 56 and/or channel 102 until it is time to inject material 130 into a body or structure, such as, for example, a body of a human patient. In some embodiments, cap 128 is screwed onto nozzle 98 by mating threads of nozzle 98 and cap 128. In some embodiments, cap 128 can be variously connected with nozzle 98, such as, for example, monolithic, integral connection, frictional engagement, threaded engagement, mutual grooves, screws, adhesive, nails, barbs and/or raised element.

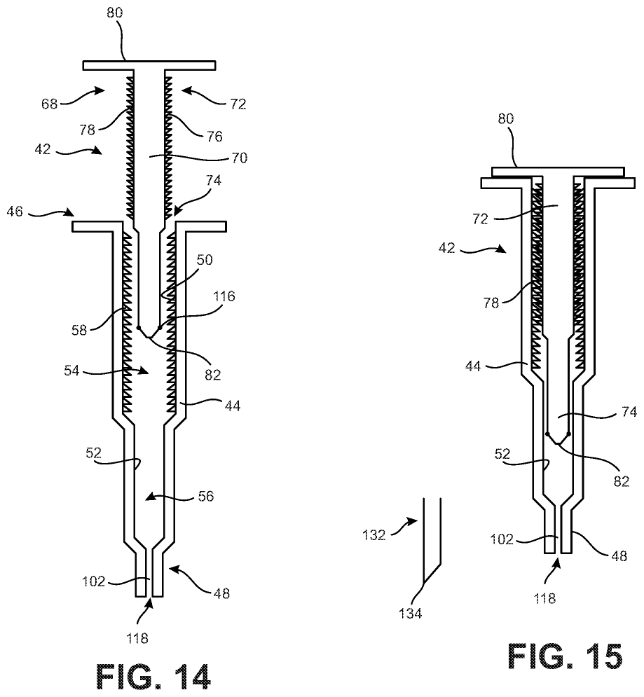

To expel material 130 from syringe 42, plunger 68 is inserted into barrel 44 by inserting section 74 into chamber 54 such that teeth 76 are spaced apart from teeth 58, as shown in FIG. 14. Cap 128 may be removed from nozzle 98 prior to inserting plunger 68 into barrel 44. Plunger 68 is translated along axis X1 in the direction shown by arrow O in FIG. 1 until teeth 76 engage teeth 58, as shown in FIG. 1. At this point, the engagement of teeth 76 with teeth 58 prevents plunger 68 from moving relative to barrel 44 in the direction shown by arrow P in FIG. 1 along axis X1. In some embodiments, the displacement of piston 68 in chamber 54 and/or chamber 56 may cause some of material 130 to exit syringe 42 through opening 118. To expel the remaining material 130 in chamber 56 and/or channel 102, plunger 68 is further translated along axis X1 in the direction shown by arrow O in FIG. 1 until plunger 68 is fully inserted into barrel 44, as shown in FIG. 15. Once material 130 is expelled from syringe 42, syringe 42 can be discarded since syringe 42 is unable to be reused.

In some embodiments, a hollow needle 132 may be coupled to a distal end of nozzle 98 to adapt syringe 42 for hypodermic uses wherein a cannula of needle 132 is in communication with channel 102. For example, needle 132 may include a sharp tip 134 configured to penetrate the skin of a patient so that material 130 can be inserted below the skin of the patient. In some embodiments, needle 132 can be variously connected with nozzle 98, such as, for example, monolithic, integral connection, frictional engagement, threaded engagement, mutual grooves, screws, adhesive, nails, barbs and/or raised element.

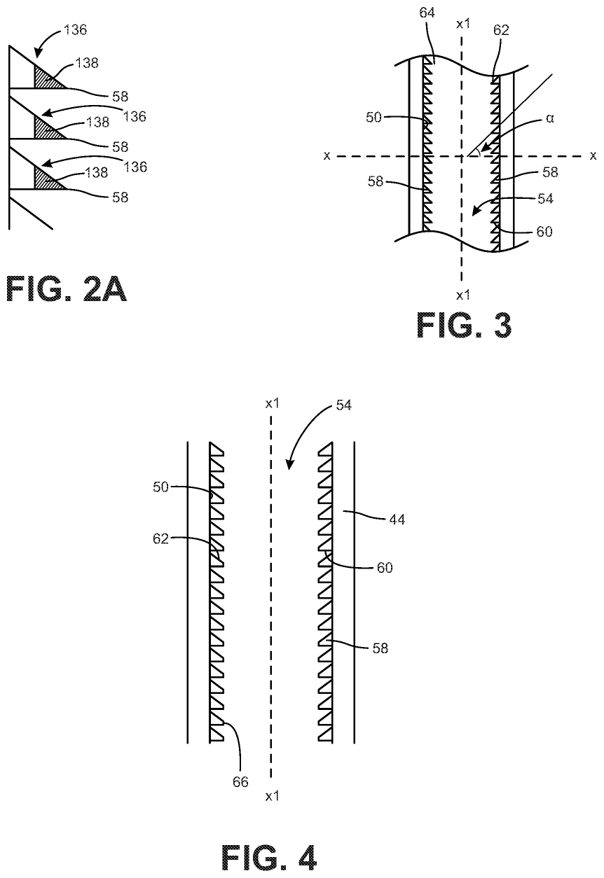

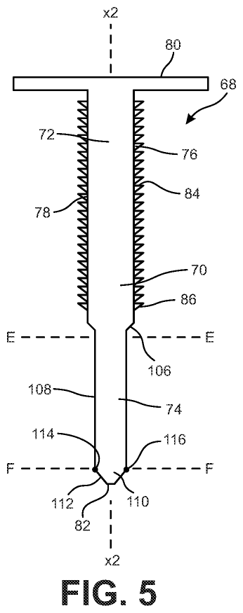

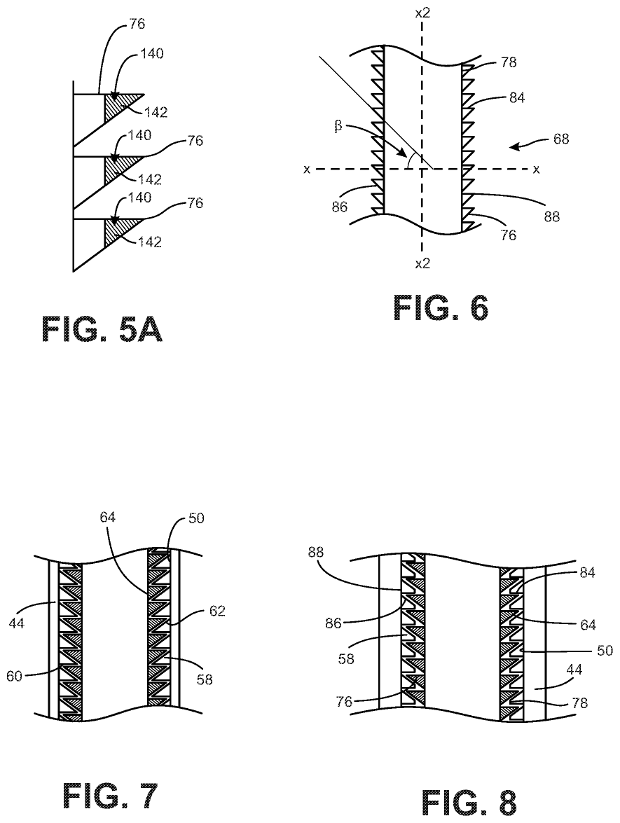

In some embodiments, teeth 58 include a pocket 136 that is filled with a material 138, such as, for example, an epoxy, as shown in FIG. 2A, for example. Pockets 136 are configured to break open as teeth 76 move over teeth 58 such that pockets 136 release material 138 from pockets 136 such that material 138 bonds with piston 68 to prevent movement of piston 68 relative to barrel 44 along axis X1 in opposite directions. In some embodiments, teeth 76 include a pocket 140 that is filled with a material 142, such as, for example, an epoxy, as shown in FIG. 5A, for example. Pockets 140 are configured to break open as teeth 58 move over teeth 76 such that pockets 140 release material 142 from pockets 140 such that material 142 bonds with barrel 44 to prevent movement of piston 68 relative to barrel 44 along axis X1 in opposite directions. In some embodiments, material 138 is a first component of an epoxy and material 142 is a second component of an epoxy, wherein combining materials 138, 142 activates the epoxy. That is, as teeth 76 move over teeth 58, materials 138, 142 are released from pockets 136, 140 such that materials 138, 142 come into contact with one another and/or combine to form the epoxy, which bonds piston 76 with barrel 44 to prevent movement of piston 68 relative to barrel 44 along axis X1 in opposite directions.

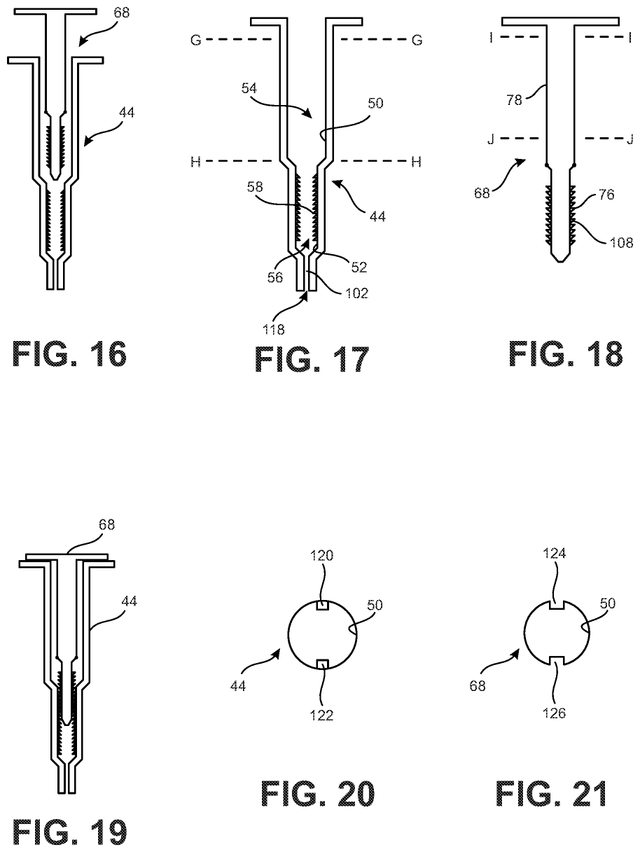

In one embodiment, shown in FIGS. 16-21, teeth 58 of barrel 44 extend from inner surface 52 rather than inner surface 50 and teeth 76 of piston 76 extend from outer surface 108 rather than outer surface 78. Furthermore, in embodiments of syringe 42 that include rails 120, 122 and grooves 124, 126, rails 120, 122 each extend outwardly from surface 50 rather than surface 52 and grooves 124, 126 each extend into outer surface 78 rather than outer surface 108.

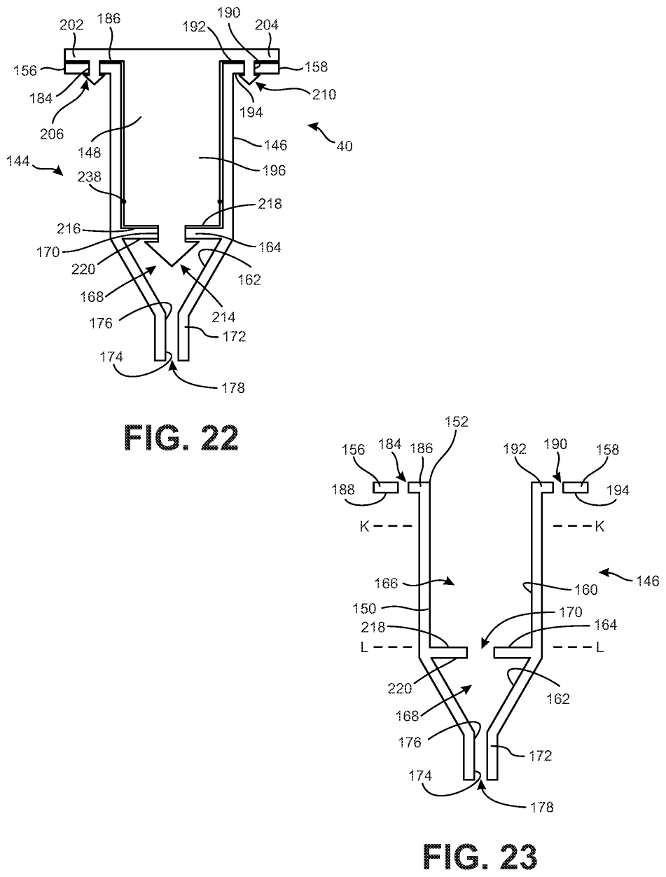

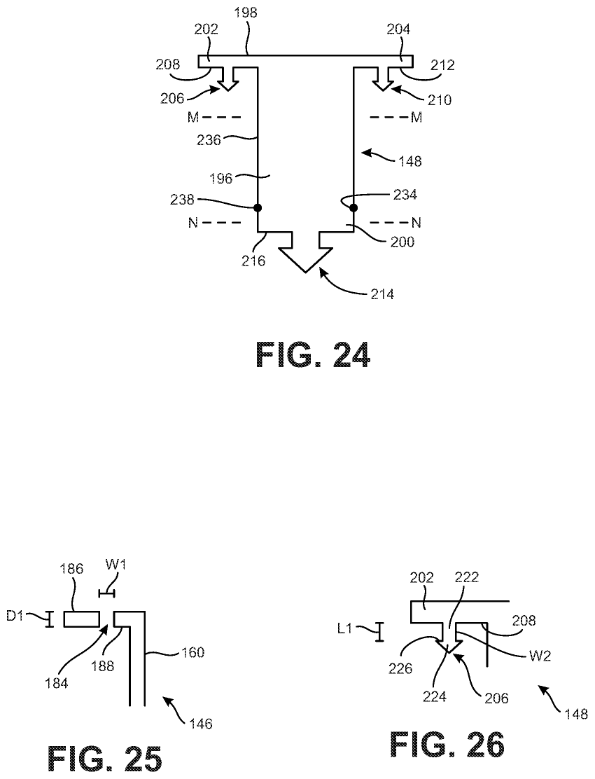

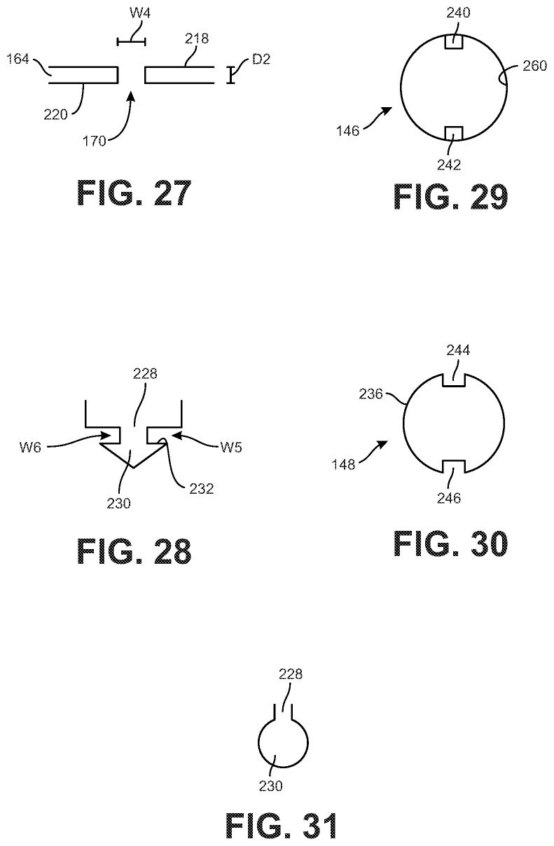

In one embodiment, shown in FIGS. 22-31, system 40 includes a syringe 144 that includes a barrel 146 and a plunger, such as, for example, a piston 148 configured to be inserted into barrel 146, as discussed herein. Barrel 146 includes a body 150. Body 150 extends along a longitudinal axis X3 between a proximal end 152 and an opposite distal end 154. Barrel 146 includes first and second arms 156, 158 extending from proximal end 152. Body 150 includes a proximal inner surface 160 and a distal inner surface 162 that is spaced apart from proximal inner surface 160 by a circumferential flange 164. Proximal inner surface 160 defines a proximal chamber 166 and distal inner surface 162 defines a distal chamber 168. Chamber 166 is in communication with chamber 168 via a central opening 170 in flange 164. In some embodiments, opening 170 extends parallel to axis X3 such that a center of opening 170 is coaxial with axis X3. In some embodiments, opening 170 may be disposed at alternate orientations, relative to axis X3, such as, for example, transverse, perpendicular and/or other angular orientations such as acute or obtuse, co-axial and/or may be offset or staggered. In some embodiments, barrel 146 is monolithic. In some embodiments, piston 148 is monolithic. In some embodiments, chamber 166 may have various cross section configurations, such as, for example, oval, oblong, triangular, rectangular, square, polygonal, irregular, uniform, non-uniform, variable, tubular and/or tapered.

In some embodiments, barrel 146 includes a nozzle 172 extending from distal end 154. Nozzle 172 includes an inner surface 174 that is continuous with inner surface 162. Inner surface 174 defines a channel 176 that is in communication with chamber 168 and an aperture 178 that is in communication with channel 176 such that a material, such as, for example, a liquid in chamber 166, chamber 168 and/or channel 176 can be expelled from syringe 144 via aperture 178, as discussed herein. In some embodiments, barrel 146 includes a hollow needle 180 coupled to a distal end of nozzle 172. In some embodiments, needle 180 includes a sharp tip 182 to allow needle 180 to pierce tissue, such as, for example, skin when syringe 144 is used to deliver a material hypodermically, for example. In some embodiments, needle 180 can be variously connected with nozzle 172, such as, for example, monolithic, integral connection, frictional engagement, threaded engagement, mutual grooves, screws, adhesive, nails, barbs and/or raised element.

Barrel 146 includes features that engage features of piston 148 when piston 148 is fully inserted into barrel 146 to prevent syringe 144 from being reused. For example, flange 164 and opening 170 are configured for engagement and/or disposal of one or more features of piston 148, as discussed herein. In some embodiments, barrel 146 includes a hole 184 extending through a proximal surface 186 of arm 156 and an opposite distal surface 188 of arm 156 and a hole 190 extending through a proximal surface 192 of arm 158 and an opposite distal surface 194 of arm 158. Holes 184, 190 and surfaces 186, 188, 192, 194 are configured for disposal and/or engagement with features of piston 148 when piston 148 is fully inserted into barrel 146 to prevent syringe 144 from being reused, as discussed herein.

Piston 148 includes a body 196 extending along a longitudinal axis X4 between a proximal end 198 and an opposite distal end 200. Body 196 is configured for disposal in chamber 166. Piston 148 includes first and second arms 202, 204 extending from proximal end 198. Piston 148 includes a prong 206 extending from a distal surface 208 of arm 202 and a prong 210 extending from a distal surface 212 of arm 204. Prong 206 is configured for disposal in hole 184 and prong 210 is configured for disposal in hole 190 when piston 148 is fully inserted into barrel 146 to prevent syringe 144 from being reused, as discussed herein. Piston 148 includes an extension 214 that extends from a distal end surface 216 of distal end 200. Extension 214 is configured for disposal and/or engagement with opening 170 and/or opposite proximal and distal surfaces 218, 220 of flange 164 when piston 148 is fully inserted into barrel 146 to prevent syringe 144 from being reused, as discussed herein. In some embodiments, body 196 may have various cross section configurations, such as, for example, oval, oblong, triangular, rectangular, square, polygonal, irregular, uniform, non-uniform, variable, tubular and/or tapered.

Prongs 206, 210 each include a shaft 222 and a barb 224 that extends from a distal end of shaft 222. Holes 184, 190 each have a width W1 that is slightly greater than a width W2 of shafts 222 to permit shafts 222 to be inserted into holes 184, 190. Barbs 224 each have a maximum width W3 that is greater than width W1 and/or width W2 such that barbs 224 must deform when moving through hole 184 or hole 190. Holes 184, 190 each have a depth D1 that is substantially equal to lengths L1 of shafts 222 such that a proximal surface 226 of barb 224 engages distal surface 188 when prong 206 is disposed in hole 184 and distal surface 212 when prong 210 is disposed in hole 190.

Extension 214 includes a shaft 228 and a barb 230 that extends from a distal end of shaft 228. Opening 170 has a width W4 that is slightly greater than a width W5 of shaft 228 to permit shaft 228 to be inserted into opening. Barb 230 has a maximum width W6 that is greater than width W4 and/or width W5 such that barb 230 must deform when moving through opening 170. Opening 170 has a depth D2 that is substantially equal to a length L2 of shaft 228 such that a proximal surface 232 of barb 230 engages distal surface 220 when shaft 228 is disposed in opening 170.

In some embodiments, surface 160 is smooth and/or even along an entire length of surface 160. That is, surface 160 is free of any cavities or recesses that extend into surface 160 and is also free of any projections or extensions that extend from surface 160. Piston 148 includes a groove 234 extending into an outer surface 236 of body 196 and a gasket, such as, for example, an O-ring 238 positioned in groove 234. O-ring 238 is configured to engage and slide along surface 160 when piston 148 is being inserted into barrel 146 such that O-ring 238 forms an airtight and/or watertight seal with surface 160. This allows section end surface 216 to push any material, such as, for example, a liquid within chamber 166 out of syringe 144 through aperture 178. In some embodiments, O-ring 238 is made from an elastomeric material.

In some embodiments, piston 148 is prevented from rotating relative to barrel 146 about axis X3 to align prongs 206, 210 with holes 184, 190 and to align extension 214 with opening 170 as piston 148 is being inserted into barrel 146. For example, in some embodiments, barrel 146 includes spaced apart first and second rails 240, 242 extending from inner surface 160 and body 196 includes spaced apart grooves 244, 246 extending into outer surface 236. Rails 240, 242 each extend parallel to axis X3 and grooves 244, 246 each extend parallel to axis X4. Rail 240 is disposed in groove 244 and rail 242 is disposed in groove 246 as piston 148 is inserted into barrel 146 to prevent piston 148 from rotating relative to barrel 146 about axis X3. In some embodiments, a portion of barrel 146 that includes chamber 166 includes a non-circular cross-sectional configuration and body 196 includes a non-circular cross-sectional configuration to prevent piston 148 from rotating relative to barrel 146 about axis X3 when body 196 is inserted into chamber 166.

In operation and use, syringe 144 may be used to mix and/or dispense a material, such as, for example, material 130. In some embodiments, material 130 comprises a material, such as, for example, a liquid, gel, paste, cement, gum, ointment, cream and/or foam. In some embodiments, material 130 is pre-loaded into at least one of chamber 166, chamber 168 and channel 176. In some embodiments, cap 128 is coupled to nozzle 172 to maintain material 130 in chamber 166, chamber 168 and/or channel 176 until it is time to inject material 130 into a body or structure, such as, for example, a body of a human patient.

To expel material 130 from syringe 144, piston 148 is inserted into barrel 146 by inserting body 196 into chamber 166. Cap 128 may be removed from nozzle 172 prior to inserting piston 148 into barrel 146. Piston 148 is translated along axis X3 in the direction shown by arrow Q in FIG. 22 until distal end surface 216 engages proximal surface 164, extension 214 extends through opening 170, prong 206 is disposed in hole 184 and prong 210 is disposed in hole 190, as discussed herein, to prevent piston 148 from moving relative to barrel 146 in the direction shown by arrow R in FIG. 22 along axis X3. In some embodiments, the displacement of body 196 in chamber 166 causes material 130 to exit syringe 144 through aperture 178. Once material 130 is expelled from syringe 144, syringe 144 can be discarded since syringe 144 is unable to be reused.

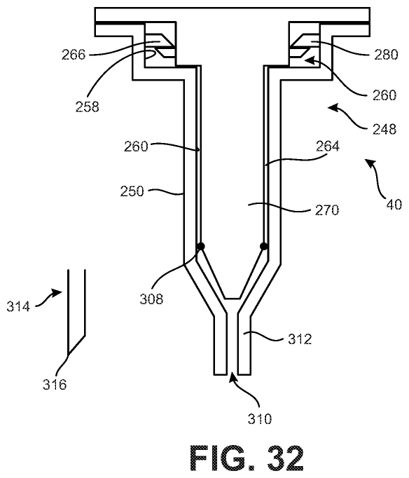

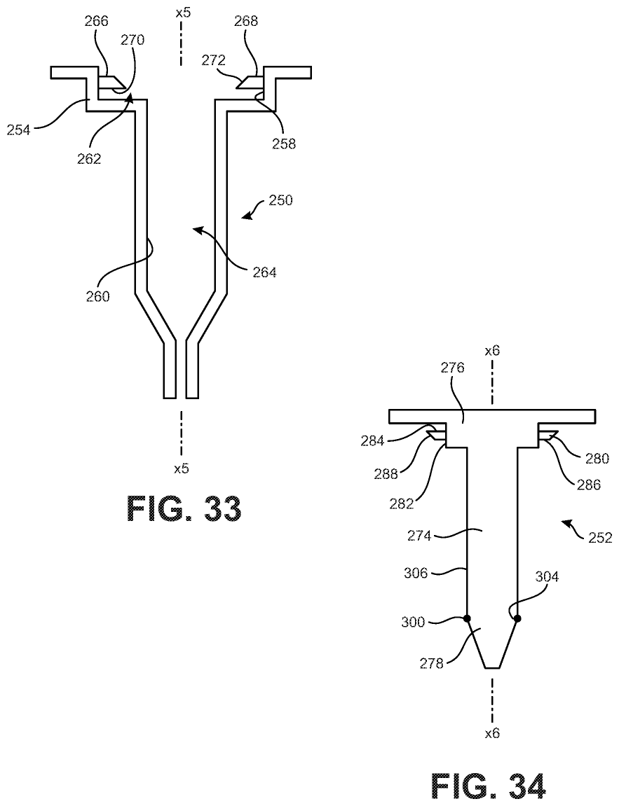

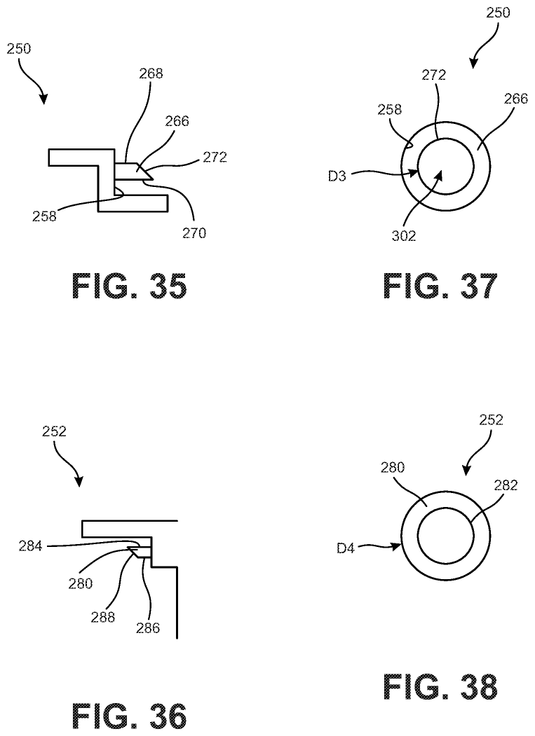

In one embodiment, shown in FIGS. 32-38, system 40 includes a syringe 248. Syringe 248 includes a barrel 250 and a piston 252 configured for disposal in barrel 250, as discussed herein. Barrel 250 extends along a longitudinal axis X5 between a proximal end 254 and an opposite distal end 256. Barrel 250 comprises a proximal inner surface 258 and a distal inner surface 260. Proximal inner surface 258 defines a proximal chamber 262 and distal inner surface 260 defines a distal chamber 264 that is in communication with chamber 262. Barrel 250 comprises a first circumferential flange 250 extending from proximal inner surface 258. First flange 266 comprises opposite proximal and distal surfaces 268, 270 and a first end surface 272 extending from proximal surface 268 to distal surface 270. In some embodiments, barrel 250 is monolithic. In some embodiments, piston 252 is monolithic. In some embodiments, chamber 264 may have various cross section configurations, such as, for example, oval, oblong, triangular, rectangular, square, polygonal, irregular, uniform, non-uniform, variable, tubular and/or tapered.

Piston 252 comprises a body 274 extending along a longitudinal axis X6 between a proximal section 276 and an opposite distal section 278. Piston 252 comprises a second circumferential flange 280 extending from an outer surface 282 of proximal section 276. Flange 280 comprises opposite proximal and distal surfaces 284, 286 and a second end surface 288 extending from proximal surface 284 to distal surface 286. Piston 252 is configured for engagement and/or disposal with barrel 250 to move syringe 248 from a first configuration in which flange 266 is positioned between flange 280 and distal end 256 to a second configuration in which flange 280 is positioned between flange 266 and distal end 256. In some embodiments, distal surface 270 of flange 266 directly engages proximal surface 284 of flange 280 when syringe 248 is in the second configuration to prevent piston 252 from translating relative to barrel 250 along longitudinal axis X5 in the direction shown by arrow S in FIG. 32. In some embodiments, body 274 may have various cross section configurations, such as, for example, oval, oblong, triangular, rectangular, square, polygonal, irregular, uniform, non-uniform, variable, tubular and/or tapered. In some embodiments, body 274 is configured to be rotatable relative to barrel 250 about axis X5 when body 274 is disposed in chamber 264. In some embodiments, body 274 is prevented from rotating relative to barrel 250 about axis X5 when body 274 is disposed in chamber 264. For example, chamber 264 and/or body 274 may have non-circular cross-sectional configurations that prevent rotation of body 274 relative to barrel 250 about axis X5 when body 274 is disposed in chamber 264, similar to chamber 52 and body 70 in FIGS. 13A-13C. It is further envisioned that barrel 250 and piston 252 may have corresponding rails and grooves, similar to rails 120, 122 and grooves 124, 126 and/or rails 240, 242 and grooves 244, 246 to prevent rotation of body 274 relative to barrel 250 about axis X5 when body 274 is disposed in chamber 264.

In some embodiments, end surface 288 slides along end surface 272 as syringe 248 moves from the first configuration to the second configuration. In some embodiments, end surface 272 extends at a first angle relative to longitudinal axis X5 and end surface 288 extends at a second angle relative to longitudinal axis X6. In some embodiments, the first angle is between about 1 degree and about 80 degrees and the second angle is between about −1 degree and about −80 degrees. In some embodiments, the first angle is between about 30 degrees and about 60 degrees and the second angle is between about −30 degrees and about −60 degrees.

In some embodiments, barrel 250 includes first and second arms 290, 292 extending in opposite directions from proximal end 254 and piston 252 comprises a ledge 294 extending from proximal section 276. A distal surface 296 of ledge 294 engages a proximal surface 298 of arm 290 and a proximal surface 300 of arm 292 when distal surface 270 of first flange 266 directly engages proximal surface 284 of second flange 280. In some embodiments, arms 290, 292 each extend perpendicular to axis X5 and ledge 294 extends perpendicular to axis X6. In some embodiments, first flange 266 defines a circular opening 302 having a diameter D3 and second flange has a diameter D4. In some embodiments, diameter D4 is greater than diameter D3. In some embodiments, diameter D4 is equal diameter D3. In some embodiments, diameter D4 is less than diameter D3.

In some embodiments, surface 260 is smooth and/or even along an entire length of surface 260. That is, surface 260 is free of any cavities or recesses that extend into surface 260 and is also free of any projections or extensions that extend from surface 260. Piston 252 includes a groove 304 extending into an outer surface 306 of body 274 and a gasket, such as, for example, an O-ring 308 positioned in groove 304. O-ring 308 is configured to engage and slide along surface 260 when piston 252 is being inserted into barrel 250 such that O-ring 308 forms an airtight and/or watertight seal with surface 260. This allows body 274 to push any material, such as, for example, a liquid within chamber 264 out of syringe 248 through an aperture 310 of barrel 250 that is in communication with chamber 264. In some embodiments, O-ring 308 is made from an elastomeric material.

In operation and use, syringe 248 may be used to mix and/or dispense a material, such as, for example, material 130. In some embodiments, material 130 comprises a material, such as, for example, a liquid, gel, paste, cement, gum, ointment, cream and/or foam. In some embodiments, material 130 is pre-loaded into chamber 264. In some embodiments, cap 128 is coupled to a nozzle 312 of barrel 250 to maintain material 130 in chamber 264 until it is time to inject material 130 into a body or structure, such as, for example, a body of a human patient.

To expel material 130 from syringe 248, piston 252 is inserted into barrel 250 by inserting body 274 into chamber 264. Cap 128 may be removed from nozzle 312 prior to inserting piston 252 into barrel 150. Piston 252 is translated along axis X5 in the direction shown by arrow T in FIG. 32 such that surface 286 engages surface 268. Piston 252 is further translated along axis X5 in the direction shown by arrow T in FIG. 32 such that surface 288 slides along surface 272. Piston 252 is further translated along axis X5 in the direction shown by arrow T in FIG. 32 until flange 280 is positioned between flange 266 and end 312 and surface 270 engages surface 284, as discussed herein, to prevent piston 252 from moving relative to barrel 250 in the direction shown by arrow S in FIG. 32 along axis X5. In some embodiments, the displacement of body 274 in chamber 264 causes material 130 to exit syringe 248 through aperture 310. Once material 130 is expelled from syringe 248, syringe 248 can be discarded since syringe 248 is unable to be reused.

In some embodiments, barrel 250 includes a hollow needle 314 coupled to a distal end of nozzle 312. In some embodiments, needle 314 includes a sharp tip 316 to allow needle 314 to pierce tissue, such as, for example, skin when syringe 248 is used to deliver a material hypodermically, for example. In some embodiments, needle 314 can be variously connected with nozzle 312, such as, for example, monolithic, integral connection, frictional engagement, threaded engagement, mutual grooves, screws, adhesive, nails, barbs and/or raised element.

In some embodiments, a kit containing one or more components of dispensing system 40 is provided. The kit may include one or more components from one or more of the embodiments discussed herein. For example, the kit may include one or more syringes 42, one or more syringes 144 and/or one or more syringes 248. The kit may further include one or more caps 128, one or more needles, such as, for example, one or more needles 132, one or more needles 180 and/or one or more needles 316. The kit may further include one or more materials, such as, for example, material 130 and/or one or more liquid materials, wherein the liquid materials can include biologics, for example. The kit may further include instructions for using syringe 42, syringe 144 and/or syringe 248.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A syringe comprising:
a barrel extending along a longitudinal axis between opposite proximal and distal ends, the barrel comprising a proximal inner surface and a distal inner surface, the proximal inner surface defining a proximal chamber and the distal inner surface defining a distal chamber, the barrel comprising first teeth extending from the proximal inner surface; and
a piston comprising a body having opposite proximal and distal sections, the piston comprising second teeth extending from an outer surface of the proximal section, the second teeth directly engaging the first teeth to allow the piston to translate distally relative to the barrel along the longitudinal axis and prevent the piston from translating proximally relative to the barrel along the longitudinal axis.

2. The syringe recited in claim 1, wherein the first teeth each include a trailing edge that faces the distal end and a leading edge that faces away from the distal end, the leading edges each extending at an acute angle relative to the longitudinal axis, the trailing edges each extending perpendicular to the longitudinal axis.

3. The syringe recited in claim 1, wherein:
the body extends along a second longitudinal axis between a proximal end that includes the proximal section and a distal end that includes the distal section; and
the second teeth each include a trailing edge that faces the proximal end of the body and a leading edge that faces away from the proximal end of the body, the leading edges each extending at an acute angle relative to the second longitudinal axis, the trailing edges each extending perpendicular to the second longitudinal axis.

4. The syringe recited in claim 1, wherein:
the first teeth each include a trailing edge that faces the distal end and a leading edge that faces away from the distal end, the leading edges each extending at an acute angle relative to the longitudinal axis, the trailing edges each extending perpendicular to the longitudinal axis;
the body extends along a second longitudinal axis between a proximal end that includes the proximal section and a distal end that includes the distal section; and
the second teeth each include a trailing edge that faces the proximal end of the body and a leading edge that faces away from the proximal end of the body, the leading edges of the second teeth each extending at an acute angle relative to the second longitudinal axis, the trailing edges of the second teeth each extending perpendicular to the second longitudinal axis.

5. The syringe recited in claim 4, wherein the acute angles are the same.

6. The syringe recited in claim 4, wherein the acute angles are different.

7. The syringe recited in claim 1, wherein the first teeth each include a trailing edge that faces the distal end, a leading edge that faces away from the distal end and an intermediate edge between the trailing edge and the leading edge, the leading edges each extending at an acute angle relative to the longitudinal axis, the trailing edges each extending perpendicular to the longitudinal axis, the intermediate edges each extending parallel to the longitudinal axis.

8. The syringe recited in claim 1, wherein:
the body extends along a second longitudinal axis between a proximal end that includes the proximal section and a distal end that includes the distal section; and
the second teeth each include a trailing edge that faces the proximal end of the body, a leading edge that faces away from the proximal end of the body and an intermediate edge between the leading edge and the trailing edge, the leading edges each extending at an acute angle relative to the second longitudinal axis, the trailing edges each extending perpendicular to the second longitudinal axis, the intermediate edges each extending parallel to the second longitudinal axis.

9. The syringe recited in claim 1, wherein the first teeth each extend 360 degrees about the longitudinal axis.

10. The syringe recited in claim 1, wherein the second teeth each extend 360 degrees about a longitudinal axis defined by the body.

11. The syringe recited in claim 1, wherein the first teeth each include a pocket that is filled with epoxy, the pockets being configured to break as the second teeth move over the first teeth.

12. The syringe recited in claim 1, wherein the second teeth each include a pocket that is filled with epoxy, the pockets being configured to break as the first teeth move over the second teeth.

13. The syringe recited in claim 1, wherein the barrel includes spaced apart first and second rails extending from the distal inner surface, the body comprising spaced apart first and second grooves extending into an outer surface of the distal section, the rails each being disposed in one of the grooves when the second teeth engage the first teeth.

14. A syringe comprising:
a barrel extending along a longitudinal axis between opposite proximal and distal ends, the barrel comprising a proximal inner surface and a distal inner surface, the proximal inner surface defining a proximal chamber and the distal inner surface defining a distal chamber, the barrel comprising first teeth extending from the distal inner surface; and
a piston comprising a body having opposite proximal and distal sections, the piston comprising second teeth extending from an outer surface of the distal section, the second teeth directly engaging the first teeth to allow the piston to translate distally relative to the barrel along the longitudinal axis and prevent the piston from translating proximally relative to the barrel along the longitudinal axis.

15. The syringe recited in claim 14, wherein:
the first teeth each include a trailing edge that faces the distal end and a leading edge that faces away from the distal end, the leading edges each extending at an acute angle relative to the longitudinal axis, the trailing edges each extending perpendicular to the longitudinal axis;
the body extends along a second longitudinal axis between a proximal end that includes the proximal section and a distal end that includes the distal section; and
the second teeth each include a trailing edge that faces the proximal end of the body and a leading edge that faces away from the proximal end of the body, the leading edges of the second teeth each extending at an acute angle relative to the second longitudinal axis, the trailing edges of the second teeth each extending perpendicular to the second longitudinal axis.

16. The syringe recited in claim 14, wherein the first teeth each include a trailing edge that faces the distal end, a leading edge that faces away from the distal end and an intermediate edge between the trailing edge and the leading edge, the leading edges each extending at an acute angle relative to the longitudinal axis, the trailing edges each extending perpendicular to the longitudinal axis, the intermediate edges each extending parallel to the longitudinal axis.

17. The syringe recited in claim 14, wherein:
the body extends along a second longitudinal axis between a proximal end that includes the proximal section and a distal end that includes the distal section; and
the second teeth each include a trailing edge that faces the proximal end of the body, a leading edge that faces away from the proximal end of the body and an intermediate edge between the leading edge and the trailing edge, the leading edges each extending at an acute angle relative to the second longitudinal axis, the trailing edges each extending perpendicular to the second longitudinal axis, the intermediate edges each extending parallel to the second longitudinal axis.

18. The syringe recited in claim 14, wherein the first teeth each extend 360 degrees about the longitudinal axis.

19. The syringe recited in claim 14, wherein the second teeth each extend 360 degrees about a longitudinal axis defined by the body.

20. A syringe comprising:
a barrel extending along a longitudinal axis between opposite proximal and distal ends, the barrel comprising a proximal inner surface and a distal inner surface, the proximal inner surface defining a proximal chamber and the distal inner surface defining a distal chamber, the barrel comprising first teeth extending from the proximal inner surface, the first teeth extending 360 degrees about the longitudinal axis, the barrel including spaced apart first and second rails extending from the distal inner surface; and
a piston comprising a body having opposite proximal and distal sections, the piston comprising second teeth extending from an outer surface of the proximal section, the second teeth directly engaging the first teeth to allow the piston to translate distally relative to the barrel along the longitudinal axis and prevent the piston from translating proximally relative to the barrel along the longitudinal axis, the body comprising spaced apart first and second grooves extending into an outer surface of the distal section, the rails each being disposed in one of the grooves when the second teeth engage the first teeth,
wherein the first teeth each include a trailing edge that faces the distal end and a leading edge that faces away from the distal end, the leading edges each extending at an acute angle relative to the longitudinal axis, the trailing edges each extending perpendicular to the longitudinal axis;
wherein the body extends along a second longitudinal axis between a proximal end that includes the proximal section and a distal end that includes the distal section, the second teeth each extending 360 degrees about the second longitudinal axis,
wherein the second teeth each include a trailing edge that faces the proximal end of the body and a leading edge that faces away from the proximal end of the body, the leading edges of the second teeth each extending at an acute angle relative to the second longitudinal axis, the trailing edges of the second teeth each extending perpendicular to the second longitudinal axis,
wherein the first teeth each include an intermediate edge between the trailing edges of the first teeth and the leading edges of the first teeth, the intermediate edges of the first teeth each extending parallel to the longitudinal axis, and
wherein the second teeth each include an intermediate edge between the leading edges of the second teeth and the trailing edges of the second teeth, the intermediate edges of the second teeth each extending parallel to the second longitudinal axis.
